(19) United States
(12) Patent Application Publication
(10) Pub. No.: US 2014/0220123 A1
(43) Pub. Date: Aug. 7, 2014

(54) PHARMACEUTICAL COMPOSITIONS COMPRISING NIACIN AND A PROCESS FOR THEIR PREPARATION

(71) Applicants: Benoit Schmitt, Irvine, CA (US); Laurent Imler, Irvine, CA (US)

(72) Inventors: Benoit Schmitt, Irvine, CA (US); Laurent Imler, Irvine, CA (US)

(73) Assignee: PHARMA PASS II LLC, Irvine, CA (US)

(21) Appl. No.: 13/756,588

(22) Filed: Feb. 1, 2013

(51) Int. Cl. A61K 9/20; A61K 9/28; A61J 3/10; A61K 31/455

(52) U.S. Cl. CPC A61K 9/2095; A61K 31/455; A61K 9/28; A61J 3/10
USPC 424/465; 514/356; 264/115; 427/2.14

(57) ABSTRACT

A process for the manufacture of compositions containing niacin, the process including a first compaction step of niacin or a pharmaceutically acceptable salt thereof, then a second compaction step of a dry blend comprising niacin and microcrystalline cellulose and a further compression step into tablets. Stable tablet compositions including niacin are capable of extended release of niacin.

### FIELD OF THE INVENTION

The present invention relates to a novel process for the manufacture of compositions containing niacin and to stable tablet compositions comprising niacin capable of extended release of niacin.

### BACKGROUND

Niacin (nicotinic acid, also known as 3-pyridinecarboxylic acid, chemical formula C6H5NO2) is a well-known and important dietary supplement. It can be represented by the following chemical formula:

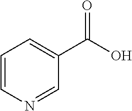

Niacin is known to play an important role in metabolism, acting as a hydrogen and electron transfer agent in carbohydrate metabolism. Furthermore, niacin forms part of nicotinamide adenine dinucleotide (NAD) and nicotinamide adenine dinucleotide phosphate (NADP), which are important intercellular carriers of reducing electrons in the electron transport system in living organisms. Moreover, niacin containing coenzymes participate in a variety of biological reactions, e.g., lipid catabolism and oxidative deamination. Niacin deficiency has been identified as the leading cause of a medical malady known as pellagra. Niacin is used as a therapeutic agent in the treatment of several diseases: It is known to have benefits associated with the treatment of hypercholesterolemia because it increases levels of high-density lipoproteins (HDL) and lowers levels of total serum cholesterol low-density lipoproteins (LDL) and triglycerides. In addition, niacin exhibits adipose tissue lipolysis, reduces plasma free fatty acid levels and decreases very low density lipoprotein synthesis. Furthermore, niacin has demonstrated efficiency in preventing manifestations of arteriosclerotic heart disease.

However, widespread use of niacin is limited due to the high incidence of “flush” that often occurs with the higher doses of niacin needed for effective lipid treatment. Flushing is a term generally used to describe niacin-induced vasodilatation. An individual experiencing flushing will develop a visible, uncomfortable hot or flushed feeling upon administration of niacin. Certain materials and/or formulations have been suggested for avoiding or reducing cutaneous flushing (U.S. Pat. No. 4,956,252, U.S. Pat. No. 5,023,245, U.S. Pat. No. 5,126,145, U.S. Pat. No. 6,090,830, US-7,179,486, US-6,080,428, US-6,129,930, US-6,406,715, US-6,469,035U.S. Pat. No. 6,676,967, U.S. Pat. No. 7,011,848, US2008/0050429, US2009/0069275, US2009/0130208, US2009/0042952, US2009/0069389), however, none has proved entirely satisfying and side-effects remains a problem for wide scale utilization of niacin products.

Further, the commercial NIASPAN(R) formulations including a release retarding agent prove difficult to produce with regular specifications. Therefore, there is a need in the pharmaceutical industry for an extended-release nicotinic acid formulation that provides reduced levels of cutaneous flushing over existing niacin formulations, while also allowing for a reproducible manufacturing process characterized by improved physical, chemical and mechanical properties.

### SUMMARY

The invention relates to a process for the preparation of tablets comprising a first compaction step of pure niacin or a pharmaceutically acceptable salt thereof, then a second compaction step of a dry blend comprising niacin and hydroxypropyl cellulose and a further compression step into tablets. Preferred embodiments comprise one or more of the following features:

The invention is directed to a process for the preparation of tablets comprising:
(i) a first compaction step of niacin or a pharmaceutically acceptable salt thereof, then:
(ii) a second compaction step of a dry blend comprising compacted niacin from step (i) and hydroxypropylcellulose and:
(iii) a further compression step into tablets.

According to an embodiment, the first compacted blend is granulated. According to an embodiment, the second compacted blend is granulated and the resulting granules are compressed into tablets. According to an embodiment, additional excipients are added to the blend of compacted niacin and hydroxypropylcellulose before the second compaction step (ii). According to an embodiment, the additional excipients are selected among binders, diluents, lubricants and glidants. According to an embodiment, the additional excipients are selected among colloidal silicon dioxide, sodium stearyl fumarate.

According to an embodiment, the tablets are further coated. According to an embodiment, the compaction steps are performed using a roll compactor. According to an embodiment, the compression step is performed using a rotating press. According to an embodiment, the first compaction step is performed at a pressure within the range of 1 to 6 Mpa. According to an embodiment, the second compaction step is performed at a pressure within the range of 1 to 6 Mpa.

The invention is directed to a tablet obtainable by the above described process. According to an embodiment, the tablet comprises 200 to 1000 mg of niacin or of a pharmaceutically acceptable salt thereof. According to an embodiment, the tablet further comprises a binder and/or a diluent and/or a glidant and/or a lubricant. According to an embodiment, the additional excipients are selected among colloidal silicon dioxide, sodium stearyl fumarate.

According to an embodiment, the tablet has the following composition wherein percentages are by weight of the tablet:

| Niacin Granules | Niacin | 200-1000.0 mg |
|---|---|---|
| Compacted Blend | Hydroxypropyl Cellulose | 10-20% |
| | Sodium stearyl fumarate | 0.1-0.5% |
| | Colloidal silicon dioxide | 0.10-0.30% |
| Compressed Blend | Sodium stearyl fumarate | 0.40-0.55% |
| | Aerosil ® | 0.30-0.45% |
| Coating Layer | Opadry ® II | 3-6% |
| Total | | 100% |

According to an embodiment, the tablet has the following composition:

| | | | |
|---|---|---|---|
| Niacin Granules | Niacin | 1000.0 | mg |
| Compacted Blend | Hydroxypropyl Cellulose | 215.0 | mg |
| | Sodium stearyl fumarate | 5.1 | mg |
| | Colloidal silicon dioxide | 2.5 | mg |
| Compressed Blend | Sodium stearyl fumarate | 6.1 | mg |
| | Aerosil ® | 4.9 | mg |
| Coating Layer | Opadry ® II | 60.0 | mg |
| Total | | 1293.60 | mg |

The invention is directed to a method for treating or preventing hypercholesterolemia, comprising the step of administering orally to a patient in need thereof, a tablet as above disclosed.

### BRIEF DESCRIPTION OF THE DRAWINGS

FIGS. 1 to 3 are schematic illustrations of the following process steps respectively: Compaction step 1 and compaction step 2, compression, cosmetic coating application; and

FIGS. 4 to 6 are graphic representations of the results of the dissolving test applied to various tablet compositions according to the invention.

### DETAILED DESCRIPTION

The present invention relies on the surprising finding that tablet compositions of niacin having an improved extended release could be obtained from a dry process comprising a first compaction step of niacin or a pharmaceutically acceptable salt thereof, then a second compaction step of a dry blend comprising compacted niacin and hydroxypropylcellulose and a further compression step into tablets. Wet granulation processes are usually preferred over dry compression techniques for the preparation of tablets. For example when the amount of an active substance contained in the tablet is low one may foresee difficulties in dispersing the drug uniformly in the powder blend using a conventional direct compression process. In addition, wet granulation processes are usually preferred for the preparation of chewable tablets. For example, the dispersion or the size of the granules obtained through the amount of wetting agent involved in the granulation usually influences the organoleptic qualities of the final product. Thus, dry techniques are more challenging as it seems more difficult to meet all the characteristics of free-flowing mixtures of the dry blend and of homogeneity. In the present case, a dry compaction process has notably the advantage over any conventional wet granulation process that the use of water and/or other wetting agents is avoided. It is a faster process as there isn't any drying step. It also saves energy as there is no heating step involved.

The process according to the invention may be performed by a first compaction step of niacin or a pharmaceutically acceptable salt thereof. Preferably, the first granulation by compaction step is performed on the pure active principle, i.e. the active principle deprived of additives. Niacin or its pharmaceutically acceptable salt is compacted into a slug or a sheet which can be subsequently sieved into compacted granules. This step has proved to be particularly advantageous to improve the flowability of the second compaction blend and thus the speed of the second compaction step.

The second compaction step comprises blending a first composition containing niacin together with a controlling agent like hydroxypropyl cellulose and optionally with other excipients. For example, the use of diluents may be advantageous to increase the bulk of the solid pharmaceutical composition. Excipients include in a non limiting way, binders, diluents, glidants, lubricants. The resulting blend is then compacted into a slug or a sheet which can be subsequently sieved into compacted granules.

The compaction step includes a passage through a rotating granulator, which results in granules of selected size. One appropriate sieving method involves passing a powder through a mesh of defined size in order to exclude particles below the specified size. Air may concomitantly be used to carry away the fine particles.

During the first compaction step, wherein only niacin or its salt are treated, the mesh is advantageously selected between 30 and 12 Mesh. During the second compaction step, wherein at least niacin granules and hydroxypropyl cellulose are blended, the mesh is advantageously selected between 30 and 12 Mesh. The operating conditions for compaction at the first and second compaction steps will be those that are available to the man skilled in the art. Within the scope of the present invention, the blends can be compacted by either slugging or passing the material between two counter-rotating rolls. The compaction force may be adjusted using a method appropriate for the compactor employed, for example by control of the rate of feed into the compactor. Advantageously, during the first compaction step, the pressure is adjusted to a value between 1 and 6 MPa. Advantageously, during the second compaction step, the pressure is adjusted to a value between 1 and 6 MPa.

The compacted granules thus obtained may subsequently be compressed into a tablet, typically with the addition of a lubricant or of other excipients. The tablets preferably have a crushing strength in the range of 50 to 300 N, for example 100 to 250 N. These can be determined by standard techniques on a Erweka Multicheck tester.

The compressed tablet may further be coated with a coating agent to form film coated tablets. The coating agent may be suspended in a solution or directly taken from a commercially available coating solution which is sprayed onto the compressed pellets. Preferably, all process steps are carried out in controlled atmosphere, such as low moisture, oxygen, temperature and light protection.

The present invention is also concerned with the resulting tablets thus obtained. These exhibit an extended release as shown in the dissolution tests performed in the examples below. The specific method of preparation is characterized by a first compaction and a second compaction steps which are performed before any compression step. The inventors have noted that the first compaction step, resulting in niacin granules, improves the flowability of the dry blend before the second compaction and therefore facilitates the preparation of tablets.

In the tablets of the present invention, niacin can be in its acid form or any of its pharmaceutically acceptable salts, solvates or polymorphs. Typically, niacin may be used in the form of an alkali metal salt, such as its sodium salt. Each tablet advantageously comprises 200 to 1000 mg niacin or a pharmaceutically acceptable salt thereof.

In the framework of this invention, binders refer to excipients which enhance the linkage between particles. They include in a non limiting manner, any of acacia, alginic acid, carbomer, sodium carboxymethylcellulose, dextrin, ethylcellulose, gelatine, glucose, guar gum, hydroxypropylcellulose, maltose, methylcellulose, povidone, polyvinylpyrrolidone, starch, hydroxyethylcellulose or polyethylene oxide. Typically, the amount of binder within the scope of the invention is comprised, based on the total amount of the tablet, within the scope of 0 to 20% by weight, for example 0 to 10% by weight, or 0 to 5% by weight. Advantageously, the tablets according to the invention are prepared without any binder, apart from the hydroxypropyl cellulose.

Hydroxypropylcellulose represents advantageously from 5 to 30% by weight of the total weight of the tablet, preferably, from 10 to 20% by weight. Different grades of hydroxypropylcellulose may be used, notably hydroxypropyl cellulose with varied molecular weights (and consequently varied viscosity) can be used. For example hydroxypropyl cellulose with molecular weight between 100 000 and 5.10⁶ may be used. Preferably, hydroxypropyl cellulose is selected from: a mixture of hydroxylpropyl cellulose of molecular weight between 300 000 and 400 000, and hydroxylpropyl cellulose of molecular weight between 700 000 and 1.10⁶.

Such components are commercially available, like:

Klucel® GXF of molecular weight 370000, and viscosity 150-400 centipoise (2% solution)

Klucel® MXF of molecular weight 850000, and viscosity 4000-6500 centipoise (2% solution).

Diluents intend to increase the bulk of the composition in order to facilitate the processing of tablets comprising low amounts of active ingredients such as in the present case with low therapeutical amounts of niacin. Diluents within the scope of the invention comprise in addition to microcrystalline cellulose, calcium, phosphate or sulfate carbonates, dextrates, dextrins, dextrose excipients, fructose, kaolin, lactitol, anhydrous lactose, lactose monohydrate, maltose, mannitol, sorbitol, sucrose, starch, pregelatinized starch, or talc. Typically, the diluent represent about 0 to about 90% by weight based on the total weight of the tablet, for example 0 to 70% by weight, i.e. about 40% by weight. Advantageously, the tablets according to the invention are prepared without any diluents.

Lubricants are usually useful to prevent adhesion during the preparation process. These are of particular use for the process of the invention which implies the preliminary compaction of powders and/or granules before the final compression into a tablet. Suitable lubricants are, in a non limiting manner, calcium stearate, glyceryl behenate, magnesium stearate, mineral oil, polyethylene glycol, sodium stearyl fumarate, stearic acid, talc, vegetal oil, sodium lauryl sulfate or zinc stearate. Lubricants may advantageously be incorporated into the composition of the tablets of the invention in an amount comprised within the range of 0.1 to 5% by weight based on the total weight of the tablet, for example 1 to 3% by weight, i.e. about 2%.

Glidants may be useful in the early stages of the process of the invention in order to improve the flowability of the powder/granules before the compaction step. Thus, glidants may come into the composition of the tablets of the invention. Suitable glidants within the scope of the invention are, in a non limiting manner, colloidal silicon dioxide, magnesium trisilicate, starch, talc or tribasic calcium phosphate. Glidants may advantageously be incorporated into the composition of the tablets of the invention in an amount comprised within the range of 0.1 to 5% by weight based on the total weight of the tablet, for example 1 to 3% by weight.

The process advantageously comprises at least one coating step. The coating agent can be made from any commercially available powder mix for preparing coating suspensions. Examples of such powders or mix are Eudragit® available from the company Evonik, which comprises hypromellose. Other additives, like colorant and pigments may be used in the coating composition, like titanium dioxide and iron oxide for example. The coating may be prepared from the individual elements or from commercially available preparation mixtures.

Coating agents may advantageously represent 0.1 to 5% by weight based on the total weight of the tablet, for example 1 to 5% by weight, i.e. 3% by weight. Further excipients are disclosed in Handbook of Pharmaceutical excipients, 2^{nd} Ed., 1994, American Pharmaceutical Association, Washington, ISBN 0 91730 66 8, by Wade A., Weller P J.). A polishing step may be comprised in the process according to the invention. In conformity with the skilled professional methods, this polishing step can comprise the application of a solution of polyethylene glycol in water and/or in alcohol, like a water/ethanol solution.

### EXAMPLES

### Example 1

### Preparation of Tablets According to the Invention

Tablets having the following composition were prepared.

| | | | |
|---|---|---|---|
| Niacin Granules | Niacin | 1000.0 | mg |
| Compacted Blend | Hydroxypropyl Cellulose | 215.0 | mg |
| | Sodium stearyl fumarate | 5.1 | mg |
| | Colloidal silicon dioxide | 2.5 | mg |
| Compressed Blend | Sodium stearyl fumarate | 6.1 | mg |
| | Aerosil ® | 4.9 | mg |
| Coating Layer | Opadry ® II | 60.0 | mg |
| Total | | 1293.60 | mg |

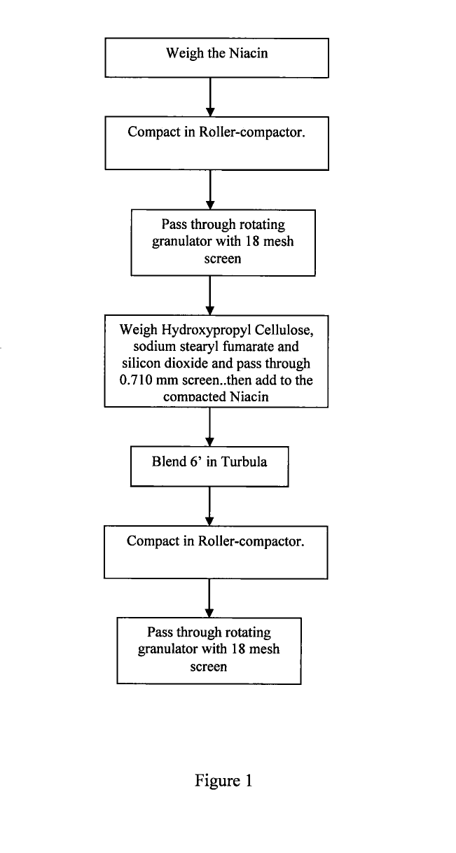

As depicted on FIG. 1, first the amount of niacin is compacted on a roller compactor TFC Labo, Vector Corp before being passed through a Vector Corp. rotating granulator (at 160 rpm) with 18 mesh screen. The roll applied a pressure of 3 Mpa at a roll speed of 3-4 rpm and a feeder screw speed of 35-36 rpm. As depicted on FIG. 1, the granules thus obtained and hydroxypropyl cellulose were mixed together with sodium stearyl fumarate and colloidal silicon dioxide for 6 minutes in Turbula T2C before being compacted on a roller compactor TFC Labo, Vector Corp. The roll applied a pressure of 4 Mpa at a roll speed of 3-4 rpm and and a feeder screw speed of 35-36 rpm.

The resulting compacted sheet was passed through a Vector Corp. rotating granulator (at 160 rpm) with 18 mesh screen. After having weighted the appropriate amount of the compacted granules for the final composition, sodium stearyl fumarate, and colloidal silica (Aerosil) were added to the mixture and blended for 5 minutes in the Turbula.

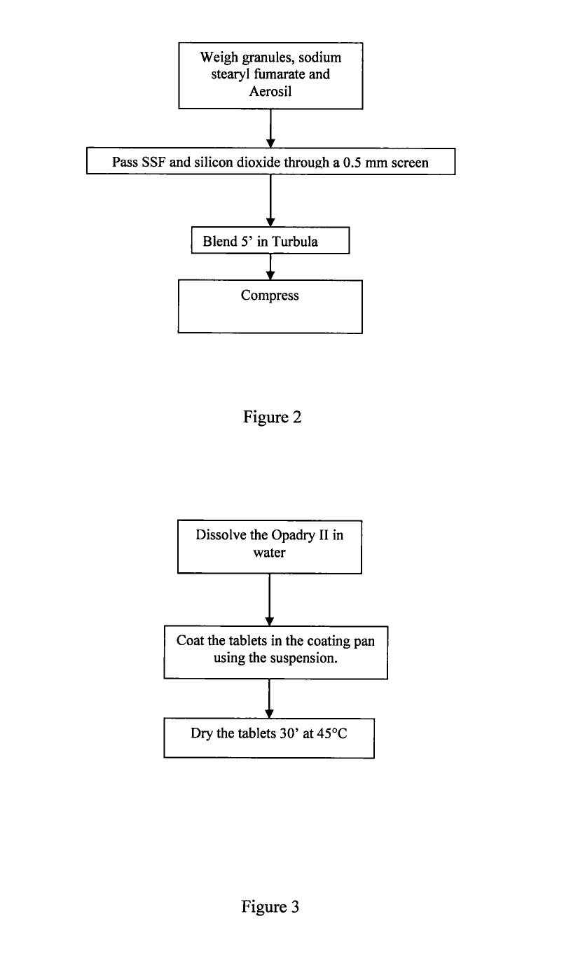

As depicted on FIG. 2, the resulting mix was finally compressed on a Manesty Betapress type rotating press using a 19.0×9.7 mm oblong tooling. The resulting tablets showed a thickness of about 7.5-7.7 mm and a hardness of 19-25 kP using a hardness tester Schleuniger 4M.

As depicted on FIG. 3, the 1234 mg tablets were further coated with a cosmetic coating by dissolving a ready to mix one-step film coating system which combines polymer, plasticizer and pigment (as available under the name Opadry® II by Colorcon®). The resulting suspension was then applied to the tablet using a 1 mm nozzle (distance nozzle-tablet bed: 8 cm; inlet temperature 50-60° C.; product temperature 38-43° C.; air flow 55-65° cfm; solution flow 5-8 g/min; spraying pressure 20-25 PSi and pan speed: 12-17 rpm) and dried for 30 minutes at 45° C.

### Example 2

### Preparation of Tablets According to the Invention and Measuring their Dissolution Properties

Further tablets were prepared by following the same method steps and comprising the ingredients disclosed in table 2 here-under. Some tablets have only been submitted to the compaction and compression steps.

| Core | A | | B | C | D | E | F | G | |
|---|---|---|---|---|---|---|---|---|---|
| Niacin, USP | 1000.00 | mg | 1000.00 mg | 1000.00 mg | 1000.00 mg | 1000.00 mg | 1000.00 mg | 100.00 | mg |
| Sodium Stearyl | 5.10 | mg | 5.10 mg | 5.10 mg | 5.10 mg | 5.10 mg | 6.20 mg | 5.10 | mg |
| Fumarate, NF. | | | | | | | | | |
| Aerosil 200 | 2.50 | mg | 2.50 mg | 2.50 mg | 2.50 mg | 2.50 mg | 2.90 mg | 2.50 | mg |
| Klucel MXF | 161.25 | mg | 215.00 mg | | 107.50 mg | 142.50 mg | 180.00 mg | 161.25 | mg |
| Klucel GXF | 53.75 | mgf | | 215.00 mg | 107.50 mg | 47.50 mg | 60.00 mg | 53.75 | mgf |
| Sodium Stearyl | 6.10 | mg | 6.10 mg | 6.10 mg | 6.10 mg | 6.0 mg | 6.30 mg | 6.10 | mg |
| Fumarate, NF. | | | | | | | | | |
| Aerosil 200 | 4.90 | mg | 4.90 mg | 4.90 mg | 4.90 mg | 4.80 mg | 5.00 mg | 4.90 | mg |
| Cosmetic | | | | | | | | | |
| Coating | | | | | | | | | |
| Opadry II | | | | | | | | 60.00 | mg |
| Purified Water | | | | | | | | 240.00 | mg |
| USP | | | | | | | | | |

The following materials have been used: Niacin was bought from Lonza Colloidal silicon dioxide was bought from Evonik under the commercial name Aerosil® 200. Hydroxypropyl Cellulose was bought from Ashland under the commercial name Klucel® MXF. Hydroxypropyl Cellulose was bought from Ashland under the commercial name Klucel® GXF. Sodium Stearyl Fumarate was bought from JRS Pharma under the commercial name Pruv®. Cosmetic coating was bought from Colorcon under the commercial name Opadry® II.

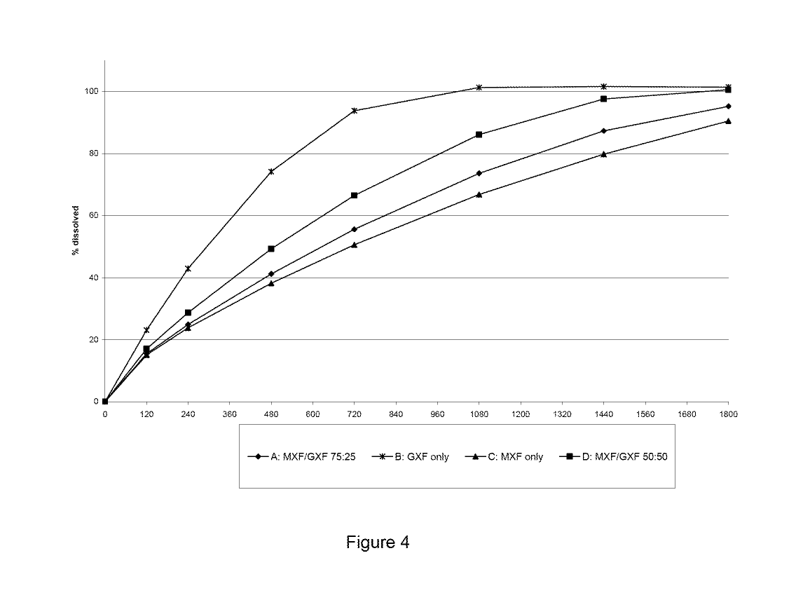

Dissolution tests: These tablet compositions have been subjected to a dissolution test in the following conditions: USP apparatus 1 (basket 10 mesh) at 100 rpm in 1000 ml purified water. The dissolution profiles are illustrated in FIGS. 4 to 6.

FIG. 4 illustrates the dissolution profile of a tablet core with different proportions of hydroxypropyl cellulose of the MXF and GXF type. Dissolution is achieved in purified water using basket 10 mesh at 100 rpm.

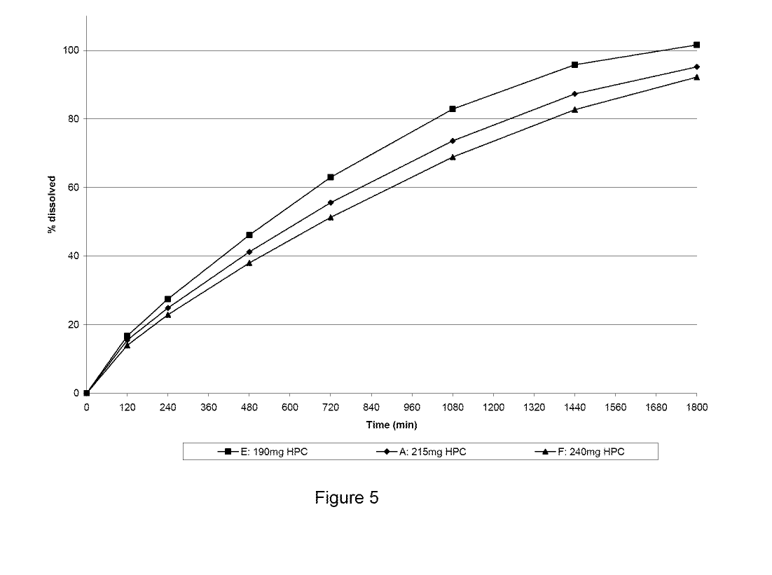

FIG. 5 illustrates the dissolution profile of a tablet core with different amounts of hydroxypropyl cellulose. Dissolution is achieved in purified water using basket 10 mesh at 100 rpm.

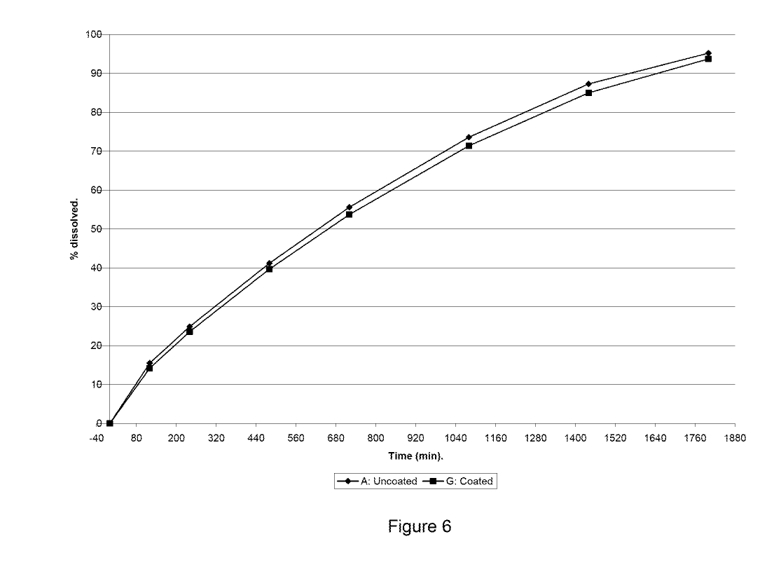

FIG. 6 illustrates the dissolution profile of a coated tablet versus uncoated tablet. Dissolution is achieved in purified water using basket 10 mesh at 100 rpm.

The dissolution profiles demonstrate that the tablets according to the invention present extended release properties.

The invention claimed is:

1. A process for the preparation of tablets comprising:
(i) a first compaction step of niacin or a pharmaceutically acceptable salt thereof; then,
(ii) a second compaction step of a dry blend comprising compacted niacin from step (i) and hydroxypropylcellulose; and
(iii) a further compression step into tablets.

2. The process according to claim 1, wherein the first compacted blend is granulated.

3. The process according to claim 1, wherein the second compacted blend is granulated and the resulting granules are compressed into tablets.

4. The process according to claim 1, wherein additional excipients are added to the blend of compacted niacin and hydroxypropylcellulose before the second compaction step (ii).

5. The process according to claim 1, wherein the additional excipients are selected among binders, diluents, lubricants and glidants.

6. The process according to claim 1, wherein the additional excipients are selected among colloidal silicon dioxide, sodium stearyl fumarate.

7. The process according to claim 1, wherein the tablets are further coated.

8. The process according to claim 1, wherein the compaction steps are performed using a roll compactor.

9. The process according to claim 1, wherein the compression step is performed using a rotating press.

10. The process according to claim 1, wherein the first compaction step is performed at a pressure within the range of 1 to 6 Mpa.

11. The process according to claim 1, wherein the second compaction step is performed at a pressure within the range of 1 to 6 Mpa.

12. A tablet obtainable by the process according to claim 1.

13. The tablet according to claim 12, comprising 200 to 1000 mg of niacin or of a pharmaceutically acceptable salt thereof.

14. The tablet according to claim 12, further comprising a binder and/or a diluent and/or a glidant and/or alubricant.

15. The tablet according to claim 14, wherein the additional excipients are selected among colloidal silicon dioxide, sodium stearyl fumarate.

16. A tablet according to claim 12, having the following composition wherein percentages are by weight of the tablet:

| Niacin Granules | Niacin | 200-1000.0 mg |
|---|---|---|
| Compacted Blend | Hydroxypropyl Cellulose | 10-20% |
| | Sodium stearyl fumarate | 0.1-0.5% |
| | Colloidal silicon dioxide | 0.10-0.30% |
| Compressed Blend | Sodium stearyl fumarate | 0.40-0.55% |
| | Aerosil ® | 0.30-0.45% |
| Coating Layer | Opadry ® II | 3-6% |
| Total | | 100% |

17. A tablet according to claim 16, having the following composition:

| | | | |
|---|---|---|---|
| Niacin Granules | Niacin | 1000.0 | mg |
| Compacted Blend | Hydroxypropyl Cellulose | 215.0 | mg |
| | Sodium stearyl fumarate | 5.1 | mg |
| | Colloidal silicon dioxide | 2.5 | mg |
| Compressed Blend | Sodium stearyl fumarate | 6.1 | mg |
| | Aerosil ® | 4.9 | mg |
| Coating Layer | Opadry ® II | 60.0 | mg |
| Total | | 1293.60 | mg |

18. A method for treating or preventing hypercholesterolemia, comprising the step of administering orally to a patient in need thereof, a tablet according to claim 12.
